(19) United States
(12) Patent Application Publication
(10) Pub. No.: US 2022/0079657 A1
(43) Pub. Date: Mar. 17, 2022

(54) ELECTRODE UNIT AND RESECTOSCOPE APPARATUS

(71) Applicant: OLYMPUS CORPORATION, Tokyo (JP)

(72) Inventors: Seiji IWASAKI, Iruma-shi (JP); Soichi IKUMA, Akishima-shi (JP)

(73) Assignee: OLYMPUS CORPORATION, Tokyo (JP)

(21) Appl. No.: 17/485,914

(22) Filed: Sep. 27, 2021

Related U.S. Application Data
(63) Continuation of application No. PCT/JP2019/014099, filed on Mar. 29, 2019.

(51) Int. Cl. A61B 18/14

(52) U.S. Cl. CPC A61B 18/149; A61B 2018/1405

(57) ABSTRACT

An electrode unit which performs treatment on tissue in a subject by using a high frequency current under observation with an endoscope includes: two electrode support members disposed in a separated manner in a direction along a predetermined axis; a base part extending along an axis intersecting with the predetermined axis, and having a distal end coupled with proximal ends of the two electrode support members; and an electrode having both ends supported by the two electrode support members, wherein the electrode includes an exposed portion in a region which overlaps with at least one of the two electrode support members when viewed from a second direction which is opposite to a first direction in which the two electrode support members are pushed against the subject during use of the electrode unit, the exposed portion being exposed only in the second direction.

### CROSS REFERENCE TO RELATED APPLICATION

This application is a continuation application of PCT/JP2019/014099 filed on Mar. 29, 2019, the entire contents of which are incorporated herein by this reference.

### BACKGROUND OF THE INVENTION

### 1. Field of the Invention

The present invention relates to an electrode unit and a resectoscope apparatus which perform treatment on tissue in a subject by using a high frequency current.

### 2. Description of the Related Art

An electrocautery is known as a device which performs treatment, such as resection or coagulation, using a high frequency current on tissue of a subject, such as a human body. Hereinafter, treatment, such as resection or coagulation, using a high frequency current is simply referred to as “treatment”. For example, Japanese Patent No. 3730796 discloses a device which performs treatment on tissue in a subject under observation with an endoscope.

In a technique disclosed in Japanese Patent No. 3730796, a high frequency current is caused to flow through an electrode formed into a loop shape to perform treatment (resection, coagulation or the like) on tissue.

The electrode formed into a loop shape, which is disclosed in Japanese Patent No. 3730796, is used for resecting the tissue in an organ, such as a bladder.

### SUMMARY OF THE INVENTION

One aspect of the present invention is directed to an electrode unit which performs treatment on tissue in a subject by using a high frequency current under observation with an endoscope, the electrode unit including: two electrode support members disposed in a separated manner in a direction along a predetermined axis; a base part extending along an axis intersecting with the predetermined axis, and having a distal end coupled with proximal ends of the two electrode support members; and an electrode having both ends supported by the two electrode support members, wherein the electrode includes an exposed portion in a region which overlaps with at least one of the two electrode support members when viewed from a second direction which is opposite to a first direction in which the two electrode support members are pushed against the subject during use of the electrode unit, the exposed portion being exposed only in the second direction.

Another aspect of the present invention is directed to an electrode unit which performs treatment on tissue in a subject by using a high frequency current under observation with an endoscope, the electrode unit including: two electrode support members disposed in a separated manner in a direction along a predetermined axis; a base part extending along an axis intersecting with the predetermined axis, and having a distal end coupled with proximal ends of the two electrode support members; and an electrode having both ends supported by the two electrode support members, wherein the electrode includes an electrode center part disposed so as to be contained within profiles of the two electrode support members when viewed from a distal end side of the two electrode support members.

Still another aspect of the present invention is directed to a resectoscope apparatus including: a sheath inserted into a subject and having a hollow shape; a telescope including an insertion portion inserted into the sheath; an electrode unit holding part configured to hold the electrode unit in the sheath; and a scope holding part configured to hold the insertion portion of the telescope in the sheath at a position in a second direction with respect to the two electrode support members of the electrode unit, the second direction being opposite to a first direction in which the two electrode support members are pushed against the subject during use of the electrode unit.

### BRIEF DESCRIPTION OF THE DRAWINGS

FIG. 1 is a view showing a schematic configuration of an endoscope system of a first embodiment;

FIG. 2 is a view showing a state where an electrode unit of the first embodiment is moved with respect to a telescope in a proximal end direction;

FIG. 3 is a view of the electrode unit of the first embodiment as viewed from a leftward direction;

FIG. 4 is a perspective view of the electrode unit of the first embodiment;

FIG. 5 is a perspective view showing electrode support parts and a treatment electrode in the first embodiment in an enlarged manner;

FIG. 6 is a view of the electrode support parts and the treatment electrode in the first embodiment as viewed from a distal end side along a first axis;

FIG. 7 is a cross-sectional view taken along VII-VII in FIG. 2;

FIG. 8 is a view showing a manner of resecting tissue by using the electrode unit of the first embodiment;

FIG. 9 is a view showing the mariner of resecting the tissue by using the electrode unit of the first embodiment;

FIG. 10 is a view showing the manner of resecting the tissue by using the electrode unit of the first embodiment;

FIG. 11 is a view showing a modification of the treatment electrode in the first embodiment;

FIG. 12 is a perspective view showing an electrode unit of a second embodiment;

FIG. 13 is a view showing a manner of resecting tissue by using the electrode unit of the second embodiment;

FIG. 14 is a view showing a modification of the electrode unit of the second embodiment;

FIG. 15 is a view showing the modification of the electrode unit of the second embodiment:

FIG. 16 is a perspective view showing an electrode unit of a third embodiment;

FIG. 17 is a view of the electrode unit of the third embodiment as viewed from a distal end side along a first axis;

FIG. 18 is a perspective view showing a first modification of the electrode unit of the third embodiment

FIG. 19 is a perspective view showing a second modification of the electrode unit of the third embodiment;

FIG. 20 is a perspective view showing a third modification of the electrode unit of the third embodiment;

FIG. 21 is a perspective view showing an electrode unit of a fourth embodiment;

FIG. 22 is a view showing a manner of resecting tissue by using the electrode unit of the fourth embodiment;

FIG. 23 is a view showing the manner of resecting the tissue by using the electrode unit of the fourth embodiment;

FIG. 24 is a perspective view showing a first modification of the electrode unit of the fourth embodiment;

FIG. 25 is a perspective view showing a second modification of the electrode unit of the fourth embodiment;

FIG. 26 is a perspective view showing a third modification of the electrode unit of the fourth embodiment;

FIG. 27 is a perspective view showing a fourth modification of the electrode unit of the fourth embodiment:

FIG. 28 is a view of an electrode unit of a fifth embodiment as viewed from a leftward direction; and

FIG. 29 is a view of the electrode unit of the fifth embodiment as viewed from an upward direction.

### DETAILED DESCRIPTION OF THE PREFERRED EMBODIMENTS

Hereinafter, preferred modes of the present invention will be described with reference to drawings. In respective drawings used in the description made hereinafter, to allow respective constitutional elements to have sizes which are visible in the drawings, each constitutional element has a different scale, and the present invention is not limited to the number of constitutional elements, the shape of the constitutional elements, size ratios of the constitutional elements, or relative positional relationships between the respective constitutional elements described in these drawings.

### First Embodiment

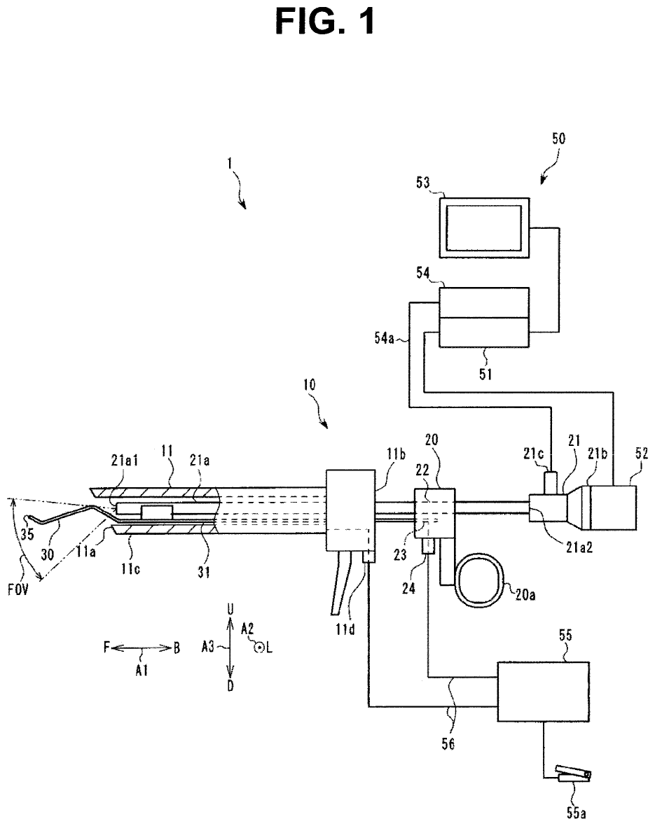

FIG. 1 is a view showing a schematic configuration of an endoscope system 1. The endoscope system 1 is a device which performs treatment (resection, coagulation or the like) on tissue in a subject by using a high frequency current under observation with an endoscope inside the subject. In the present embodiment, the subject is a human body, for example. However, the subject may be other living body.

The endoscope system 1 of the present embodiment includes a resectoscope apparatus 10 that is an endoscope, an electrode unit 30, and an external device 50. The resectoscope apparatus 10 includes a sheath 11, a slider 20, and a telescope 21.

The sheath 11 has a cylindrical hollow portion having a straight line shape along a longitudinal axis A1. The sheath 11 is a portion to be inserted into the subject from an outside of the subject when the resectoscope apparatus 10 is used. Both ends of the sheath 11 in a direction along the longitudinal axis A1 are open. During the use of the resectoscope apparatus 10, the telescope 21 and the electrode unit 30 which will be described later are inserted into the sheath 11.

The sheath 11 has a configuration for introducing perfusate into the subject. However, such a configuration is substantially equal to a corresponding configuration in a conventional resectoscope apparatus and hence, illustration and description of such a configuration will be omitted. In the present embodiment, the perfusate may be an electrolyte solution, such as saline solution, and has conductivity.

Of both ends of the sheath 11 in the direction along the longitudinal axis A1, the end on a side to be inserted into the subject is referred to as “distal end 11a”, and the end on a side opposite to the distal end 11a is referred to as “proximal end 11b”. The proximal end 11b of the sheath 11 is exposed to an outside of the subject during the use of the resectoscope apparatus 10.

Hereinafter, for the sake of description, an axis A2 and an axis A3 forming a pair are specified, the axis A2 and the axis A3 being orthogonal to the longitudinal axis A1 and being orthogonal to each other. One direction along the longitudinal axis A1 is taken as a distal end direction F, and the other direction along the longitudinal axis A1 is taken as a proximal end direction B. The distal end direction F is a direction toward the distal end 11a from the proximal end 11b of the sheath 11 along the longitudinal axis A1.

Further, in the description made hereinafter, one direction along the axis A2 is taken as a rightward direction R, and the other direction along the axis A2 is taken as a leftward direction L. One direction along the axis A3 is taken as an upward direction U, and the other direction along the axis A3 is taken as a downward direction D. In the present embodiment, a horizontal direction in an image picked up by using the telescope 21 is substantially parallel to the axis A2, and a vertical direction is substantially parallel to the axis A3. The upward direction U and the rightward direction R are on an upper side and a right side of the image picked up by using the telescope 21. In FIG. 1, the axis A2 is orthogonal to a paper surface, and a viewer's side when a viewer faces the paper surface is the leftward direction L.

A recovery electrode 11c made of a conductive material is provided to a surface of the sheath 11 at least in the vicinity of the distal end 11a. In the present embodiment, for example, the entire sheath 11 is made of a conductive material, such as metal, so that the entire sheath 11 forms the recovery electrode 11c.

A sheath connector 11d is provided in the vicinity of the proximal end 11b of the sheath 11. The sheath connector 11d is electrically connected to the recovery electrode 11c. A cable 56 is connected to the sheath connector 11d. The cable 56 electrically connects the sheath connector 11d with a high frequency power supply control device 55 of the external device 50.

The slider 20 is disposed on the proximal end 11b side of the sheath 11. The slider 20 moves relative to the sheath 11 in the direction along the longitudinal axis A1. A handle 20a is provided to the slider 20. When a user applies a force to the handle 20a with the finger, the slider 20 moves relative to the sheath 11 in the direction along the longitudinal axis A1. A mechanism of guiding the slider 20 in a movable manner relative to the sheath 11 is substantially equal to a corresponding mechanism in the conventional resectoscope apparatus and hence, illustration and description of such a mechanism will be omitted.

The slider 20 includes a scope holding part 22, an electrode unit holding part 23, and an electrode connector 24. The scope holding part 22 holds the telescope 21.

The telescope 21 is a device for optically observing the inside of the subject, The telescope 21 includes an elongated insertion portion 21a, an eyepiece part 21b, and a light source connection part 21c. The insertion portion 21a is inserted into the sheath 11 in a state where the telescope 21 is fixed by the scope holding part 22.

A distal end portion 21a1 of the insertion portion 21a is provided with an observation window and an illumination light emitting window. A proximal end portion 21a2 of the insertion portion 21a is provided with the eyepiece part 21b and the light source connection part 21c.

An image pickup unit 52 is mounted on the eyepiece part 21b. The image pickup unit 52 is electrically connected to a video processor 51 of the external device 50. An image display device 53 is electrically connected to the video processor 51. One end of an optical fiber cable 54a is connected to the light source connection part 21c. The other end of the optical fiber cable 54a is connected to a light source device 54 of the external device 50.

A field of view FOV from the observation window, which is provided to the distal end portion 21a1 of the insertion portion 21a, is picked up as an image by the image pickup unit 52, and is displayed on the image display device 53. Illumination light emitted from the light source device 54 is emitted from the illumination light emitting window provided to the distal end portion 21a1 of the insertion portion 21a. Configurations of the telescope 21 and the external device 50, which is connected to the telescope 21, are substantially equal to corresponding configurations in the conventional resectoscope apparatus and hence, detailed description of such configurations will be omitted.

The electrode unit holding part 23 holds the electrode unit 30 which will be described later. The electrode connector 24 is electrically connected to the electrode unit 30 which is held by the electrode unit holding part 23. A cable 56 is connected to the electrode connector 24. The cable 56 electrically connects the electrode connector 24 with the high frequency power supply control device 55 of the external device 50.

The electrode unit 30 includes a portion inserted through the sheath 11 in a state where the electrode unit 30 is fixed to the electrode unit holding part 23. The slider 20, together with the electrode unit 30, moves relative to the sheath 11 along the longitudinal axis A1. The electrode unit 30 is positioned in the downward direction D with respect to the telescope 21 in a state where the electrode unit 30 is fixed to the electrode unit holding part 23.

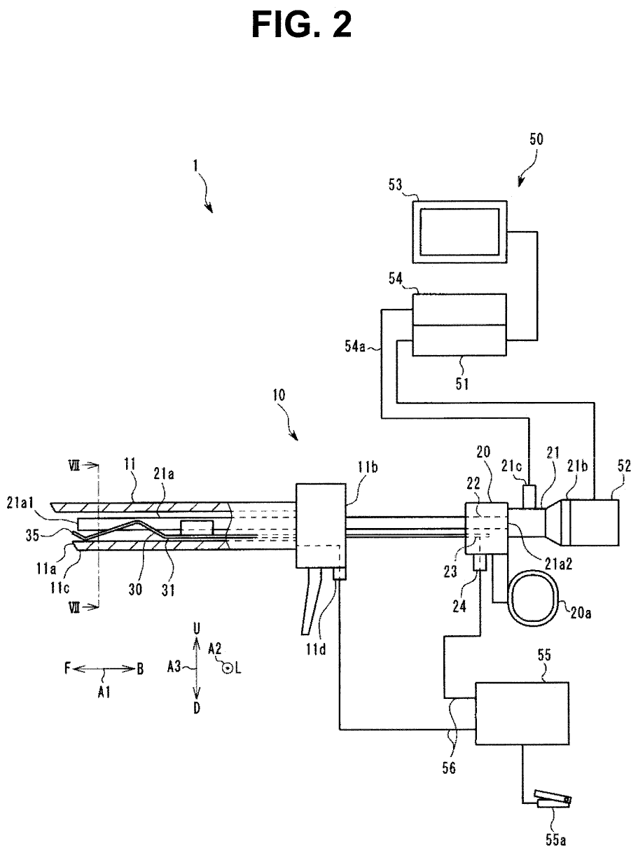

The slider 20 has a configuration of moving the electrode unit 30 relative to the telescope 21 along the longitudinal axis A1. For example, the scope holding part 22 holds the telescope 21 such that the telescope 21 is movable relative to the slider 20 along the longitudinal axis A1. FIG. 1 shows a state where the electrode unit 30 is moved with respect to the telescope 21 in the distal end direction F. FIG. 2 shows a state where the electrode unit 30 is moved with respect to the telescope 21 in the proximal end direction B.

As shown in FIG. 1, in the state where the electrode unit 30 is moved with respect to the telescope 21 in the distal end direction F, a distal end of the electrode unit 30 can be disposed at a position in the distal end direction F with respect to the distal end 11a of the sheath 11. As will be described later, a treatment electrode 35 is provided at the distal end of the electrode unit 30.

As shown in FIG. 2, in the state where the electrode unit 30 is moved with respect to the telescope 21 in the proximal end direction B, the entire electrode unit 30 can be disposed at a position in the proximal end direction B with respect to the distal end 11a of the sheath 11. That is to say, in the state where the electrode unit 30 is moved with respect to the telescope 21 in the proximal end direction B, the distal end of the electrode unit 30 is accommodated in the sheath 11. A configuration of moving the electrode unit 30 relative to the telescope 21 along the longitudinal axis A1 is substantially equal to a corresponding configuration in the conventional resectoscope apparatus and hence, the detailed description of such a configuration will be omitted.

The electrode unit 30, the recovery electrode 11c, and the high frequency power supply control device 55 constitute a so-called bipolar electrosurgical apparatus. The high frequency power supply control device 55 includes a switch 55a, a power supply circuit, and a control device.

The switch 55a may be a foot switch which is operated by the user by foot, for example. The switch 55a is a device for allowing the user to input, to the high frequency power supply control device 55, an instruction to output a high frequency current.

Based on an operation of the switch 55a performed by the user, the high frequency power supply control device 55 switches the presence or absence of the output of a high frequency current to the electrode unit 30. The high frequency current outputted from the high frequency power supply control device 55 flows among the treatment electrode 35, perfusate, and the recovery electrode 11c inside the subject. In a state where the high frequency power supply control device 55 is outputting a high frequency current, heat is generated in tissue of the subject that is brought into contact with the treatment electrode 35, thus performing treatment (resection, coagulation or the like) on the tissue.

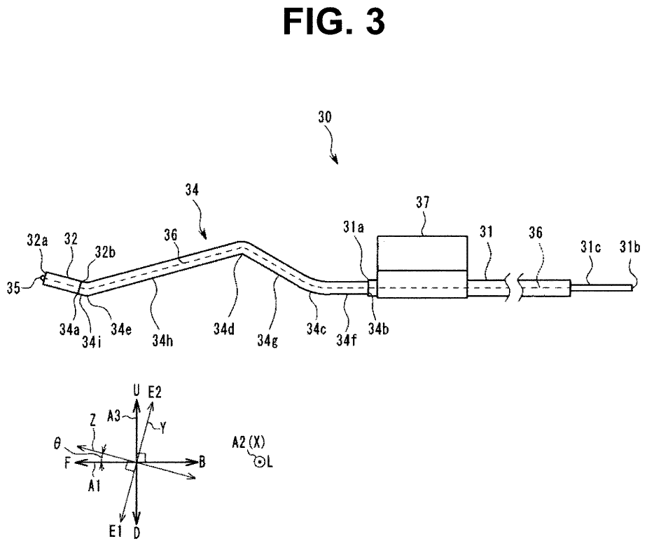

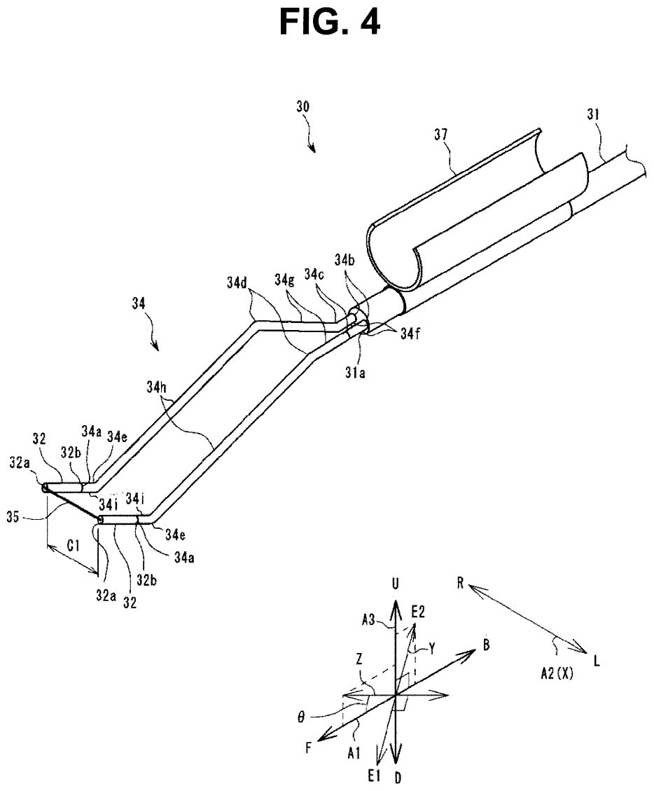

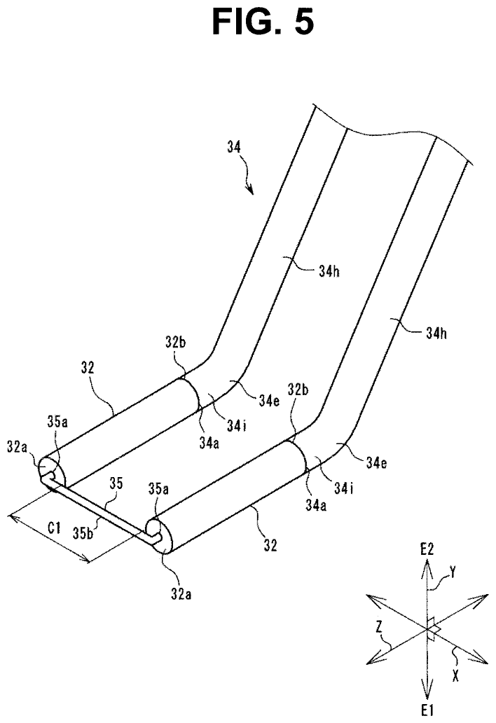

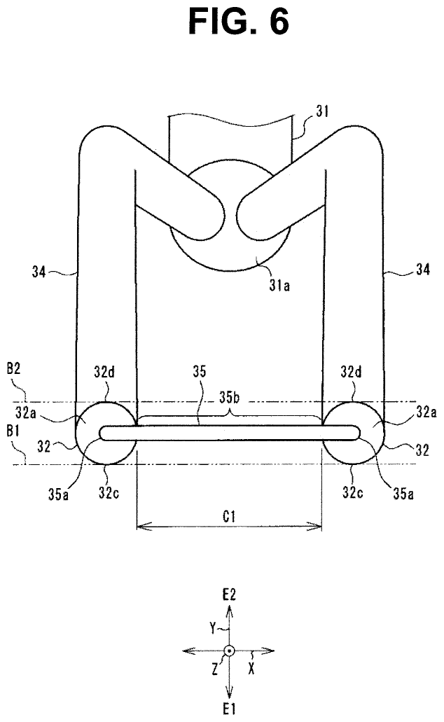

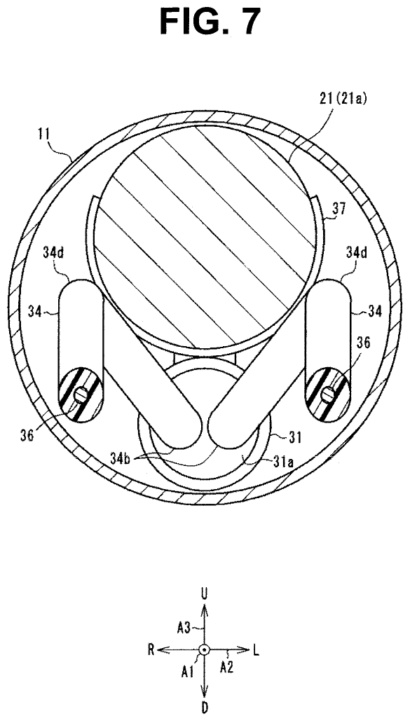

FIG. 3 is a view of the electrode unit 30 as viewed from the leftward direction L. In FIG. 3, the upper side of the drawing is the upward direction U. FIG. 4 is a perspective view of the electrode unit 30. FIG. 5 is a perspective view showing electrode support parts 32 and the treatment electrode 35 in an enlarged manner. FIG. 6 is a view of the electrode support parts 32 and the treatment electrode 35 as viewed from a distal end side along a first axis Z. FIG. 7 is a cross-sectional view taken along VII-VII in FIG. 2.

The electrode unit 30 includes a base part 31, two electrode support parts 32, and the treatment electrode 35.

As shown in FIG. 3 and FIG. 4, the base part 31 is a rod-like portion having a straight line shape. A surface of the base part 31 is covered by a material having electrical insulating property. The base part 31 is fixed to the electrode unit holding part 23 of the resectoscope apparatus 10. The electrode unit holding part 23 holds a proximal end 31b of the base part 31. The base part 31 is substantially parallel to the longitudinal axis A1 in a state where the base part 31 is fixed to the electrode unit holding part 23.

The description of the direction and the arrangement of components of the electrode unit 30 is made hereinafter assuming that the electrode unit 30 is in a state of being fixed to the electrode unit holding part 23. That is to say, in the description made hereinafter, the base part 31 is substantially parallel to the longitudinal axis A1, and the base part 31 is substantially orthogonal to the axis A2 and the axis A3.

The proximal end 31b of the base part 31 is provided with an electrical connection part 31c. The electrical connection part 31c is electrically connected to the electrode connector 24 of the resectoscope apparatus 10 in a state where the base part 31 is fixed to the electrode unit holding part 23. The electrical connection part 31c is electrically connected to the treatment electrode 35 via a conductive wire 36 which is inserted through the electrode unit 30.

A guide part 37 is provided in the vicinity of a distal end 31a of the base part 31. The guide part 37 is fitted on an outer periphery of the insertion portion 21a of the telescope 21, and slides on the insertion portion 21a in the direction along the longitudinal axis A1. In the present embodiment, the guide part 37 is a hollow member having an annular shape or a C shape in cross section.

The guide part 37 is fixed to a surface of the outer peripheral surface of the base part 31, the surface facing in the upward direction U. The guide part 37 is fitted on the insertion portion 21a to position the base part 31 at a position below the insertion portion 21a of the telescope 21 in the downward direction D. The guide part 37 slides on the insertion portion 21a in the direction along the longitudinal axis A1 to guide the base part 31 such that the base part 31 moves with respect to the insertion portion 21a of the telescope 21 along the longitudinal axis A1.

As shown in FIG. 4, FIG. 5, and FIG. 6, two electrode support parts (electrode support members) 32 are coupled with the distal end 31a of the base part 31. An outer surface of the individual electrode support part 32 is made of a material having electrical insulating property. The two electrode support parts 32 extend in a direction along the predetermined first axis Z, and are disposed in a separated manner in a direction along a second axis X orthogonal to the first axis Z.

In the present embodiment, as shown in FIG. 3 and FIG. 4, the first axis Z is arranged on a plane A1-A3 including the longitudinal axis A1 and the axis A3. The first axis Z may be parallel to the longitudinal axis A1, or may intersect with the longitudinal axis A1 at a predetermined angle. In the present embodiment, for example, the first axis Z intersects with the longitudinal axis A1 at a first angle θ. As shown in FIG. 3, the first angle θ is an acute angle at which the first axis Z intersects with the longitudinal axis A1. It is assumed that the first angle θ takes a positive value when the first axis Z extends in the upward direction U as the first axis Z extends in the distal end direction F from a point of intersection between the first axis Z and the longitudinal axis A1.

The second axis X is orthogonal to the plane A1-A3 including the longitudinal axis A1 and the axis A3. That is to say, the second axis X is parallel to the axis A2. Therefore, the two electrode support parts 32 are disposed in a separated manner in the direction along the axis A1. The two electrode support parts 32 are separated in a direction along the second axis X (the axis A2) by a predetermined distance C1. In the description made hereinafter, an axis orthogonal to the first axis Z and the second axis Xis taken as a third axis Y.

In the present embodiment, the individual electrode support part 32 has a columnar shape extending in a direction along the first axis Z. The first angle θ is a positive value. The two electrode support parts 32 are disposed at the same position when viewed in the direction along the second axis X (the axis A2).

In the description made hereinafter, an end of each electrode support part 32 in the distal end direction F is referred to as “distal end 32a”, and an end of each electrode support part 32 in the proximal end direction B is referred to as “proximal end 32b”. The first angle θ is the positive value and hence, the individual electrode support part 32 is disposed in a posture where the electrode support part 32 extends in the upward direction U as the electrode support part 32 extends toward the distal end 32a from the proximal end 32b.

In the embodiment illustrated, the individual electrode support part 32 has a columnar shape. However, a cross sectional shape of the individual electrode support part 32 is not limited to a circular shape. The cross sectional shape of the individual electrode support part 32 may be an elliptical shape or an elongated circular shape, or may be a triangular shape, a quadrangular shape or other shape, for example. Further, in the embodiment illustrated, the distal ends 32a of the electrode support parts 32 are located at positions in the upward direction U with respect to the base part 31. However, the distal ends 32a of the electrode support parts 32 may be located at positions in the downward direction D with respect to the base part 31.

The two electrode support parts 32 and the base part 31 are coupled with each other via two arm parts 34. Each arm part 34 is a rod-like member that couples the proximal end 32b of the electrode support part 32 with the distal end 31a of the base part 31. Note that the two electrode support parts 32 may be directly coupled with the base part 31.

In the description made hereinafter, an end of each arm part 34 in the distal end direction F is referred to as “distal end 34a”, and an end of each arm part 34 in the proximal end direction B is referred to as “proximal end 34b”. The distal end 34a of each arm part 34 is fixed to the proximal end 32b of each electrode support part 32, and the proximal end 34b of each arm part 34 is fixed to the distal end 31a of the base part 31.

As shown in FIG. 3, each of the two arm parts 34 includes three portions which are bent in an upward direction or a downward direction when viewed in the direction along the second axis X (the axis A2). Specifically, each of the two arm parts 34 includes a first bent part (first bent portion) 34c, a second bent part (second bent portion) 34d, and a third bent part (third bent portion) 34e in this order from the proximal end 34b toward the distal end 34a.

In the description made hereinafter, a portion of each of the two arm parts 34 which is disposed in the proximal end direction B with respect to the first bent part 34c is taken as a first section 34f. A portion of each of the two arm parts 34 which is disposed between the first bent part 34c and the second bent part 34d is taken as a second section 34g. A portion of each of the two arm parts 34 which is disposed between the second bent part 34d and the third bent part 34e is taken as a third section 34h. A portion of each of the two arm parts 34 which is disposed in the distal end direction F with respect to the third bent part 34e is taken as a fourth section 34i.

In the present embodiment, the first section 34f, the second section 34g, the third section 34h, and the fourth section 34i of each of the two arm parts 34 have a straight line shape.

Each first section 34f is fixed to the distal end 31a of the base part 31. The first section 34f is disposed substantially parallel to the longitudinal axis A1 when viewed in the direction along the second axis X (the axis A2). The first bent part 34c is a portion which is bent so as to extend in the upward direction U as the first bent part 34c extends in the distal end direction F. That is to say, when viewed in the direction along the second axis X (the axis A2), each second section 34g is disposed in an inclined manner with respect to the longitudinal axis A1 so as to extend in the upward direction U as the second section 34g extends in the distal end direction F. The first sections 34f of the two arm parts 34 are disposed in such a manner that a distance by which the first sections 34f of the two arm parts 34 are separated from each other in the direction along the second axis X (the axis A2) increases as the first sections 34f of the two arm parts 34 extend in the distal end direction F.

When viewed in the direction along the second axis X (the axis A2), each second bent part 34d is disposed in the upward direction U with respect to the distal end 31a of the base part 31. The second bent part 34d is a portion which is bent so as to extend in the downward direction D as the second bent part 34d extends in the distal end direction F. That is to say, when viewed in the direction along the second axis X (the axis A2), each third section 34h is disposed in an inclined manner with respect to the longitudinal axis A1 so as to extend in the downward direction D as the third section 34h extends in the distal end direction F.

Each third bent part 34e is a portion which is bent so as to extend in the upward direction U as the third bent part 34e extends in the distal end direction F.

Each fourth section 34i is parallel to the first axis Z. That is to say, the fourth section 34i is parallel to the electrode support part 32. The fourth section 34i is fixed to the proximal end 32b of each electrode support part 32.

As shown in FIG. 7 which is a cross-sectional view, in the present embodiment, two second bent parts 34d are disposed at a position where the sheath 11 having a cylindrical shape has the largest inner diameter, the two second bent parts 34d protruding from the base part 31 in the upward direction U, the leftward direction L. and the rightward direction R. A part of the insertion portion 21a of the telescope 21 is disposed so as to be sandwiched between the two second bent parts 34d. In the sheath 11 having a cylindrical shape, the insertion portion 21a of the telescope 21 and the two arm parts 34 are disposed as in the case of the present embodiment, so that an outer diameter of the sheath 11 can be reduced.

The treatment electrode 35 is supported by the two electrode support parts 32. The treatment electrode 35 is a linear member having conductivity, such as a metal wire. Two ends 35a of the treatment electrode 35 are respectively fixed to the two electrode support parts 32. In the present embodiment, for example, the two ends 35a of the treatment electrode 35 are fixed to end surfaces of the distal ends 32a of the two electrode support parts 32.

The treatment electrode 35 is electrically connected to the wires 36 which are inserted through the electrode support parts 32, the arm parts 34, and the base part 31. In the present embodiment, for example, the wires 36 and the treatment electrode 35 are formed of the same linear member made of metal.

As shown in FIG. 6, when viewed in the direction along the first axis Z, the treatment electrode 35 includes an electrode center part 35b located between the two electrode support parts 32. The electrode center part 35b is disposed such that a longitudinal direction of the electrode center part 35b is substantially along the second axis X (the axis A2).

In the description made hereinafter, of directions along the third axis Y, a direction substantially in the downward direction D is taken as a first direction E1, and a direction opposite to the first direction E1 is taken as a second direction E2. Therefore, the first axis Z is inclined with respect to the longitudinal axis A1 at the first angle θ in such a manner that a distance by which the first axis Z is separated from the longitudinal axis A1 in the second direction E2 increases as the first axis Z extends in the distal end direction F. The first direction E1 is a direction in which the electrode support parts 32 are pushed against the subject during the use of the electrode unit 30.

When viewed in the direction along the first axis Z (when viewed from a distal end side of the electrode support parts 32), the electrode center part 35b is disposed inside a region obtained by connecting end portions of the two electrode support parts 32. In the embodiment, the region obtained by connecting the end portions of the two electrode support parts 32 is a region sandwiched between a straight line B1 and a straight line B2, the straight line B1 being in contact with ends 32c of the two electrode support parts 32 in the first direction E1, the straight line B2 being in contact with ends 32d of the two electrode support parts 32 in the second direction E2.

When viewed in the direction along the first axis Z, the electrode center part 35b is disposed on a second direction E2 side of the straight line B1, which is in contact with the ends 32c of the two electrode support parts 32 in the first direction E1. When viewed in the direction along the first axis Z, the electrode center part 35b is disposed on a first direction E1 side of the straight line B2, which is in contact with the ends 32d of the two electrode support parts 32 in the second direction E2.

That is to say, the electrode center part 35b is disposed in such a manner that, when viewed in the direction along the first axis Z, the electrode center part 35b is prevented from protruding beyond profiles of the two electrode support parts 32 in the first direction E1. The electrode center part 35b is disposed so as to be prevented from protruding beyond the profiles of the two electrode support parts 32 in the downward direction D.

Next, the operation of the electrode unit 30 and the resectoscope apparatus 10 having the above-described configuration will be described. An example of treatment to be performed on the tissue of the subject may be resection of the tissue in an organ 100, such as the bladder.

In a case of resecting the tissue in the organ 100 by using the electrode unit 30 and the resectoscope apparatus 10, the user inserts the sheath 11 of the resectoscope apparatus 10 into the organ 100, and fills the inside of the organ 100 with perfusate. At this point of operation, the insertion portion 21a of the telescope 21 and the electrode unit 30 are accommodated in the sheath 11. A method for inserting the resectoscope apparatus 10 into the organ 100 and a method for filling the inside of the organ 100 with perfusate are substantially equal to corresponding conventional methods and hence, the description of such methods will be omitted.

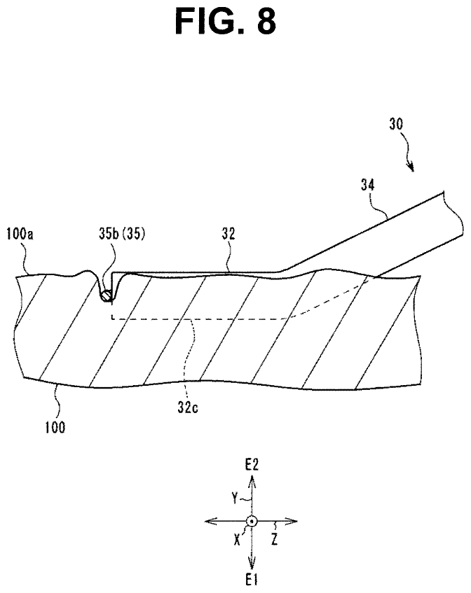

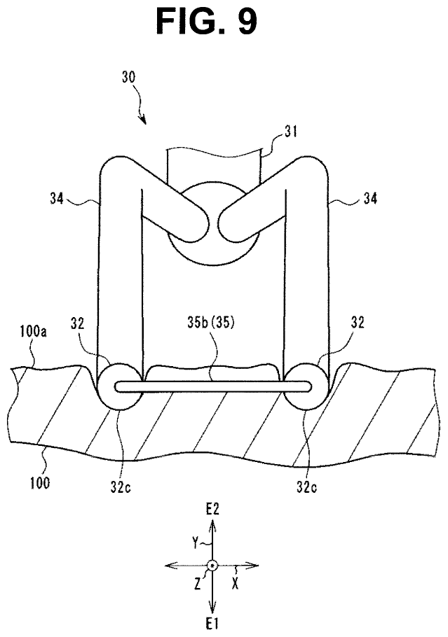

Next, the user causes the two electrode support parts 32 of the electrode unit 30 to protrude from the distal end 11a of the sheath 11 in the distal end direction F. Then, the user moves the resectoscope apparatus 10 while observing an image picked up by using the telescope 21 and, as shown in FIG. 8 and FIG. 9, pushes the two electrode support parts 32 against a wall surface 100a of the organ 100. Specifically, the user brings the ends 32c of the two electrode support parts 32 in the first direction E1 into contact with the wall surface 100a of the organ 100, and applies a force in the first direction E1 to the two electrode support parts 32.

When the ends 32c of the two electrode support parts 32 in the first direction E1 are pushed against the wall surface 100a of the organ 100, the wall surface 100a has some degree of elasticity and hence, as shown in FIG. 8 and FIG. 9, the two electrode support parts 32 sink into the wall surface 100a.

The electrode center part 35b of the treatment electrode 35 is located on the second direction E2 side of the ends 32c of the two electrode support parts 32 in the first direction E1. Therefore, a depth by which the electrode center part 35b of the treatment electrode 35 sinks into the wall surface 100a is smaller than a depth by which the two electrode support parts 32 sink into the wall surface 100a.

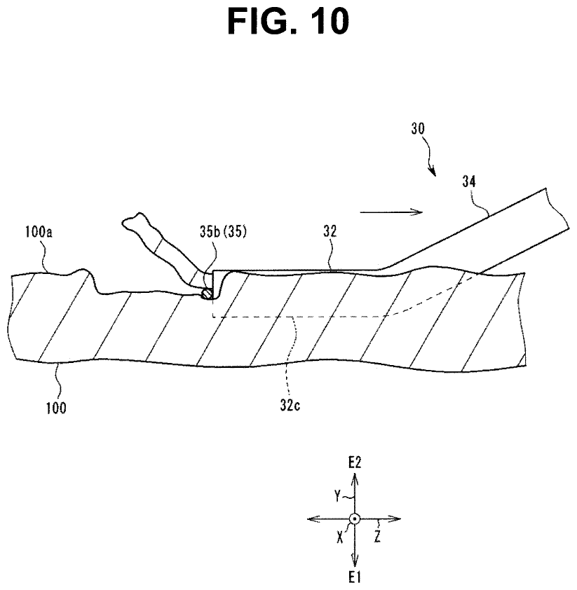

Next, the user starts an output of a high frequency current from the high frequency power supply control device 55 by operating the switch 55a. Then, as shown in FIG. 10, the user moves the two electrode support parts 32 along the wall surface 100a of the organ 100 while pushing the ends 32c of the two electrode support parts 32 in the first direction E1 against the wall surface 100a of the organ 100. That is to say, the user moves the two electrode support parts 32 in the direction along the first axis Z.

With such an operation, the electrode center part 35b of the treatment electrode 35 moves along the wall surface 100a in a state where the electrode center part 35b sinks into the wall surface 100a by a predetermined depth. At this point of operation, the tissue of a part through which the electrode center part 35b passes is cut by a high frequency current and hence, as shown in FIG. 10, the tissue of the wall surface 100a of the organ 100 is cut out with a constant thickness.

As has been described heretofore, with the electrode unit 30 and the resectoscope apparatus 10 of the present embodiment, the ends 32c of the two electrode support parts 32 in the first direction E1 are brought into contact with the tissue of the organ 100, thus maintaining a constant depth by which the electrode center part 35b sinks into the tissue. That is to say, in the electrode unit 30 and the resectoscope apparatus 10 of the present embodiment, the two electrode support parts 32 serve as stoppers that restrict a depth by which the electrode center part 35b sinks into the wall surface 100a when the electrode center part 35h is pushed against the wall surface 100a of the organ 100. Accordingly, with the electrode unit 30 and the resectoscope apparatus 10 of the present embodiment, it is possible to easily control a depth of the tissue to be treated.

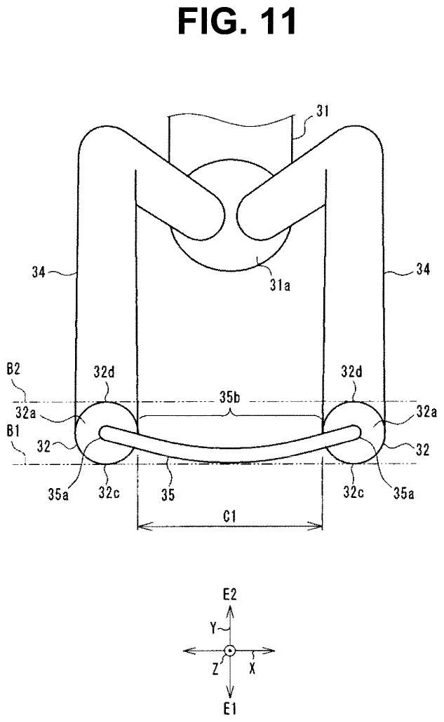

In the present embodiment shown in FIG. 6, the electrode center part 35b has a straight line shape. However, a shape of the electrode center part 35b is not limited to a straight line shape. FIG. 11 shows a modification of the electrode center part 35b. As shown in FIG. 11, the electrode center part 35b may be curved. Alternatively, the electrode center part 35b may be bent at one or a plurality of places.

### Second Embodiment

Hereinafter, a second embodiment of the present invention will be described. Hereinafter, only points which make the second embodiment different from the first embodiment will be described. Constitutional elements substantially equal to the corresponding constitutional elements in the first embodiment are given the same reference symbols, and the description of such constitutional elements will be omitted when appropriate.

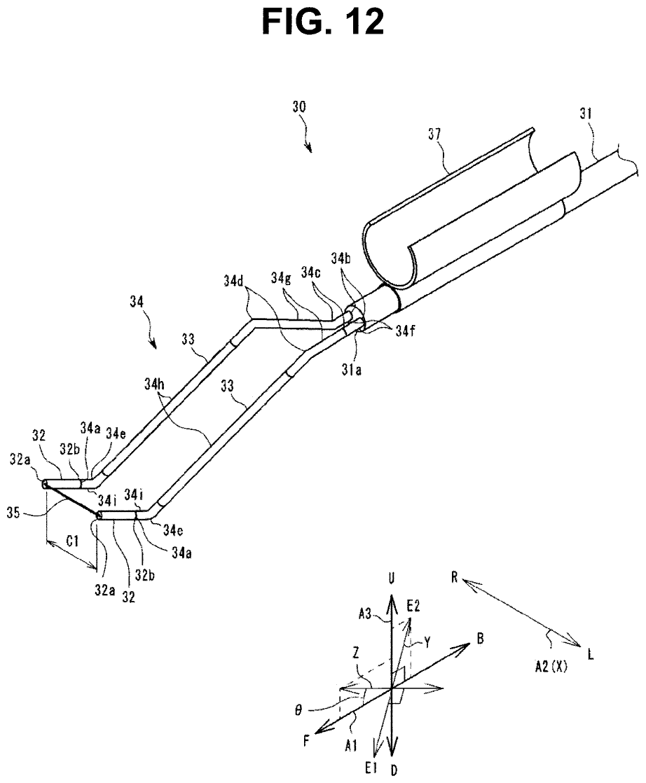

The second embodiment differs from the first embodiment with respect to a configuration of an electrode unit 30. FIG. 12 shows the electrode unit 30 of the second embodiment. The electrode unit 30 of the present embodiment includes elastic support parts 33.

The elastic support parts (elastic support members) 33 form a part or the whole of the arm parts 34, and have lower bending rigidity than the electrode support parts 32 and the base part 31. The elastic support part 33 is provided to each of two arm parts 34. In the present embodiment shown in FIG. 12, for example, each elastic support part 33 is formed by causing a part or the whole of the third section 34h of the arm part 34 to have lower bending rigidity than other sections of the arm part 34.

In the electrode unit 30 of the present embodiment, each elastic support part 33 is curved and deformed along a plane orthogonal to the second axis X (the axis A2), so that the first angle θ at which the first axis Z intersects with the longitudinal axis A1 changes. That is to say, in the electrode unit 30 of the present embodiment, an angle of the end 32c of each electrode support part 32 in the first direction E1 can be changed due to curving deformation of each elastic support part 33.

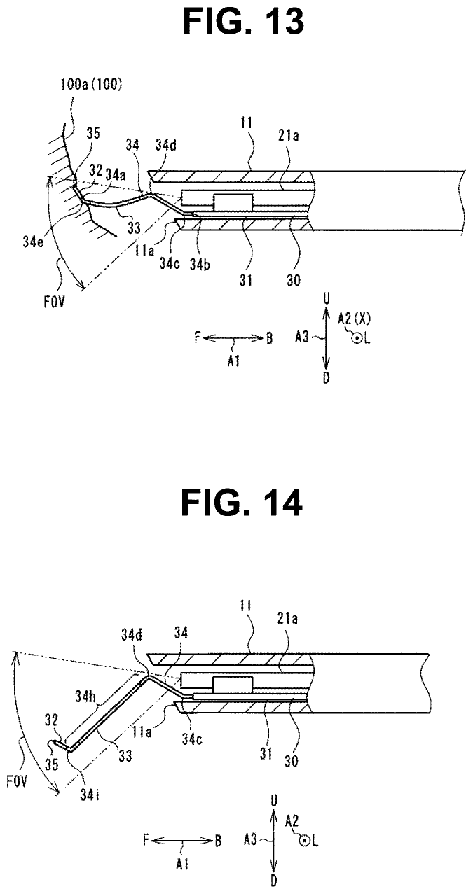

In the electrode unit 30 and the resectoscope apparatus 10 of the present embodiment, the angle of the end 32c of each electrode support part 32 in the first direction E1 can be changed due to curving deformation of each elastic support part 33 to conform to an angle of the wall surface 100a of the organ 100 with respect to the sheath 11. Therefore, in the electrode unit 30 and the resectoscope apparatus 10 of the present embodiment, for example, as shown in FIG. 13, the ends 32c of the electrode support parts 32 in the first direction E1 can also be pushed against the wall surface 100a which faces the distal end 11a of the sheath 11 and hence, treatment can be performed on tissue of the wall surface 100a.

In the present embodiment, even in a case where a force at which the electrode support parts 32 are pushed against the wall surface 100a by the user changes, the elastic support parts 33 are bent and hence, it is possible to maintain a substantially constant depth by which the electrode center part 35b sinks into the wall surface 100a. Further, even in a case where a trajectory along which the resectoscope apparatus 10 is moved by the user does not follow a shape of the wall surface 100a of the organ 100, so that a distance between the wall surface 100a and the distal end 11a of the sheath 11 changes, in the present embodiment, the elastic support parts 33 are elastically deformed, thus maintaining a state where the electrode support parts 32 are brought into contact with the wall surface 100a. When the state is brought about where the electrode support parts 32 are brought into contact with the wall surface 100a, as described above, it is possible to maintain a constant depth by which the electrode center part 35b sinks into the tissue.

As has been described heretofore, with the electrode unit 30 and the resectoscope apparatus 10 of the present embodiment, it is possible to easily control a depth of the tissue to be treated.

As described in the first embodiment, each of the two arm parts 34 of the electrode unit 30 of the present embodiment includes the first bent part 34c, the second bent part 34d, and the third bent part 34e in this order from the proximal end 34b toward the distal end 34a.

The first bent part 34c is bent in the upward direction U as the first bent part 34c extends in the distal end direction F from the proximal end 34b, which is disposed below the insertion portion 21a of the telescope 21 in the downward direction D. The second bent part 34d is located at a position overlapping with the insertion portion 21a of the telescope 21 in a direction along the axis A3 (up-and-down direction), and is bent in the downward direction D as the second bent part 34d extends in the distal end direction F. The third bent part 34e is located at a position in the downward direction D with respect to the insertion portion 21a of the telescope 21 and is bent to the first angle θ so as to extend in the upward direction U as the third bent part 34e extends in the distal end direction F.

That is to say, in the distal end direction F from the proximal end 34b of each arm part 34 coupled with the base part 31, the arm part 34 is bent in the upward direction U and, thereafter, is bent in an opposite direction (the downward direction D), and then is further bent in the upward direction U so as to have a predetermined first angle θ.

As described above, the second bent parts 34d are disposed in the sheath 11 such that the second bent parts 34d sandwiches the insertion portion 21a of the telescope 21. In general, as shown in FIG. 13, the field of view FOV of the telescope 21 in the downward direction D from the insertion portion 21a is large, but the field of view FOV of the telescope 21 in the upward direction U from the insertion portion 21a is small.

For example, unlike the present embodiment, when each arm part 34 disposed in the distal end direction F with respect to the second bent part 34d is disposed parallel to the insertion portion 21a, for example, each elastic support part 33 is located at a position substantially equal to the position of the insertion portion 21a of the telescope 21 in the up-and-down direction. In the example, when the elastic support parts 33 are curved and deformed in the upward direction U, the treatment electrode 35 moves in the upward direction U with respect to the field of view FOV.

In contrast to such an example, in the present embodiment, each second bent part 34d is caused to curve in the downward direction D as the second bent part 34d extends in the distal end direction F so as to dispose each elastic support part 33 at a position in the downward direction D with respect to the insertion portion 21a of the telescope 21. Therefore, in the electrode unit 30 and the resectoscope apparatus 10 of the present embodiment, even in a case where the elastic support parts 33 are curved and deformed at a large angle, it is possible to retain the treatment electrode 35 within the field of view FOV of the telescope 21. The case where the elastic support parts 33 are curved and deformed at a large angle means a case where the first angle θ is large and the wall surface 100a of the organ 100 faces the distal end 11a of the sheath 11.

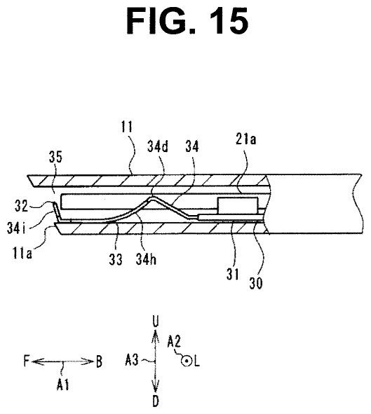

FIG. 14 and FIG. 15 show a modification of the electrode unit 30 of the present embodiment. In the electrode unit 30 of the modification, as shown in FIG. 14, in a state where portions of the electrode unit 30 disposed in the distal end direction F with respect to the second bent parts 34d protrude from the distal end 11a of the sheath 11 in the distal end direction F, the electrode support parts 32 and the treatment electrode 35 are located at positions in the downward direction D with respect to the sheath 11. Each of the portions of the electrode unit 30 disposed in the distal end direction F with respect to the second bent parts 34d includes the third section 34h and the fourth section 34i of the arm part 34, the electrode support part 32, and the treatment electrode 35.

FIG. 15 shows a state where the electrode unit 30 is moved with respect to the telescope 21 in the proximal end direction B to accommodate the treatment electrode 35 in the sheath 11. As shown in FIG. 15, in accommodating the treatment electrode 35 in the sheath 11, the elastic support parts 33 are brought into contact with an inner wall surface of the sheath 11, thus being curved and deformed and hence, it is possible to accommodate the electrode support parts 32 and the treatment electrode 35 in the sheath 11.

As in the case of the modification, in a case of performing treatment in a state where the treatment electrode 35 is caused to protrude from the sheath 11, when the treatment electrode 35 is disposed in the downward direction D with respect to the sheath 11, as shown in FIG. 14, the treatment electrode 35 can be located at a lower position within the field of view FOV of the telescope 21 in the downward direction D in a state where the elastic support parts 33 are not curved and deformed.

Therefore, in the electrode unit 30 and the resectoscope apparatus 10 of the modification, even in the case where the elastic support parts 33 are curved and deformed at a large angle, it is possible to retain the treatment electrode 35 within the field of view FOV of the telescope 21.

### Third Embodiment

Hereinafter, a third embodiment of the present invention will be described. Hereinafter, only points which make the third embodiment different from the first embodiment will be described. Constitutional elements substantially equal to the corresponding constitutional elements in the first embodiment are given the same reference symbols, and the description of such constitutional elements will be omitted when appropriate.

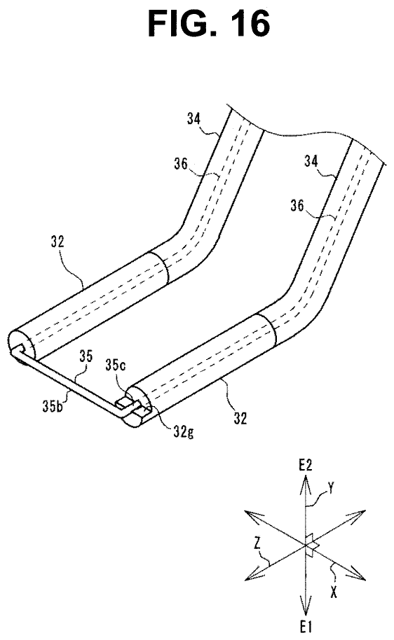

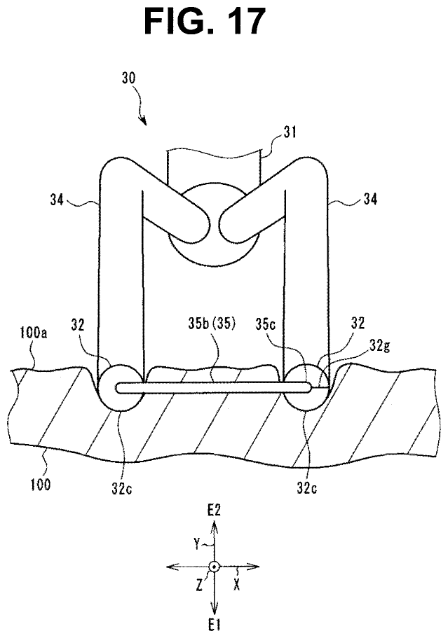

The third embodiment differs from the first embodiment with respect to the configuration of an electrode unit 30. FIG. 16 is a perspective view of electrode support parts 32 and a treatment electrode 35 of the electrode unit 30 of the third embodiment. FIG. 17 is a view of the electrode support parts 32 and the treatment electrode 35 of the electrode unit 30 of the third embodiment as viewed in the direction along the first axis Z.

The treatment electrode 35 in the present embodiment includes an exposed part 35c. A surface of the exposed part 35c is made of a, material having conductivity, and the exposed part 35c is electrically connected to the electrode center part 35b. In the present embodiment, for example, the exposed part 35c and the treatment electrode 35 are formed of the same member. Specifically, the exposed part 35c is a part of the metal wire 36 forming the treatment electrode 35. Note that the exposed part 35c may be formed of a member different from a member forming the treatment electrode 35.

The exposed part 35c is exposed in the second direction E2 in a region which overlaps with at least one of the two electrode support parts 32 when viewed from the second direction E2 side along the third axis Y. The second direction E2 is a. direction opposite to the first direction E1 in which the two electrode support parts 32 are pushed against the wall surface 100a of the organ 100. The exposed part 35c is exposed to an outer surface of the electrode unit 30, on a surface of the electrode support part 32 on a side opposite to a portion (the end 32c) which is pushed against the wall surface 100a of the organ 100.

A configuration of the exposed part 35c is not particularly limited. In the present embodiment, for example, the exposed part 35c is formed by providing a cut-out 32g extending to the wire 36 from the surface of the electrode support part 32. that faces in the second direction E2. In the present embodiment illustrated the exposed part 35c is provided to one electrode support part 32. However, the exposed part 35c may be provided to each of both electrode support parts 32.

With the electrode unit 30 and the resectoscope apparatus 10 of the present embodiment, as shown in FIG. 17, the exposed part 35c can be exposed to the inside of the organ 100 so as to come into contact with perfusate in a state Where the electrode center part 35b is caused to sink into the wall surface 100a of the organ 100.

Some high frequency power supply control devices 55 have a safety stop function that measures an electric resistance value between the treatment electrode 35 and the recovery electrode 11c and that stops an output of a high frequency current when the resistance value is higher than a predetermined value. For example, when the entire treatment electrode 35 sinks into the wall surface 100a of the organ 100, there is a possibility that an output of a high frequency current is stopped due to an action of the safety stop function.

In the electrode unit 30 and the resectoscope apparatus 10 of the present embodiment, the exposed part 35c is provided to the treatment electrode 35, so that it is possible to prevent the entire treatment electrode 35 from sinking into the wall surface 100a of the organ 100. Therefore, with the electrode unit 30 and the resectoscope apparatus 10 of the present embodiment, it is possible to prevent malfunction of the safety stop function of the high frequency power supply control device 55.

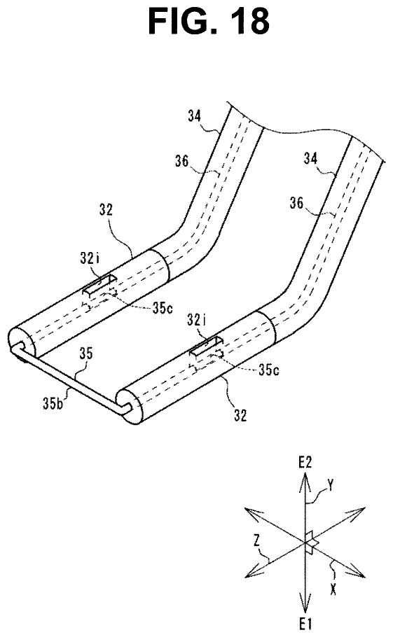

FIG. 18 shows a first modification of the exposed part 35c of the electrode unit 30 of the present embodiment. In an electrode unit 30 of the first modification shown in FIG. 18, exposed parts 35c are formed by providing window parts 32i each of which is a hole extending to the wire 36 from a surface of the electrode support part 32 that faces in the second direction E2. Note that the window part 32i may be provided to each arm part 34.

The window part 32i is open on the surface of each electrode support part 32 on the side opposite to the portion (the end 32c) which is pushed against the wall surface 100a of the organ 100. Therefore, with the electrode unit 30 of the modification, the exposed parts 35c can be exposed to the inside of the organ 100 so as to come into contact with perfusate with certainty in a state where the electrode support parts 32 and the electrode center part 35b are caused to sink into the wall surface 100a of the organ 100.

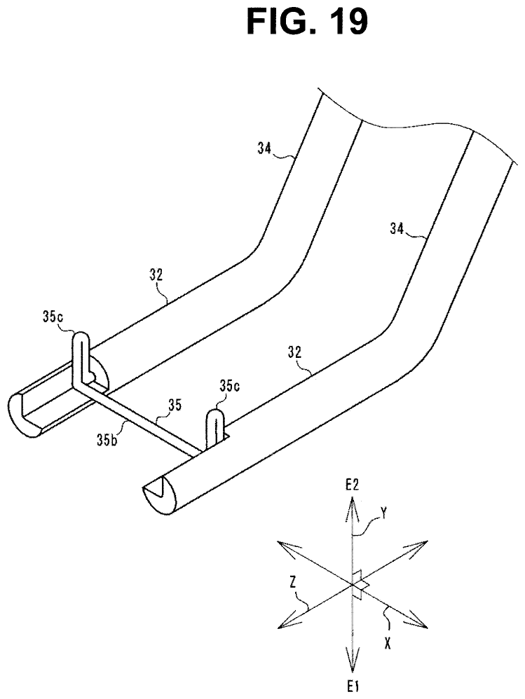

FIG. 19 shows the first modification of the exposed parts 35c of the electrode unit 30 of the present embodiment. Exposed parts 35c of a second modification shown in FIG. 19 protrude in the second direction E2 from outer surfaces of the electrode support parts 32.

Each exposed part 35c protrudes in a direction opposite to the direction in which the electrode support part 32 is pushed against the wall surface 100a, each exposed part 35c protruding from the surface of the electrode support part 32 on a side opposite to the portion (the end 32c) which is pushed against the wall surface 100a of the organ 100. With the electrode unit 30 of the modification, the exposed parts 35c can be exposed to the inside of the organ 100 so as to come into contact with perfusate with certainty in a state Where the electrode support parts 32 and the electrode center part 35b are caused to sink into the wall surface 100a of the organ 100.

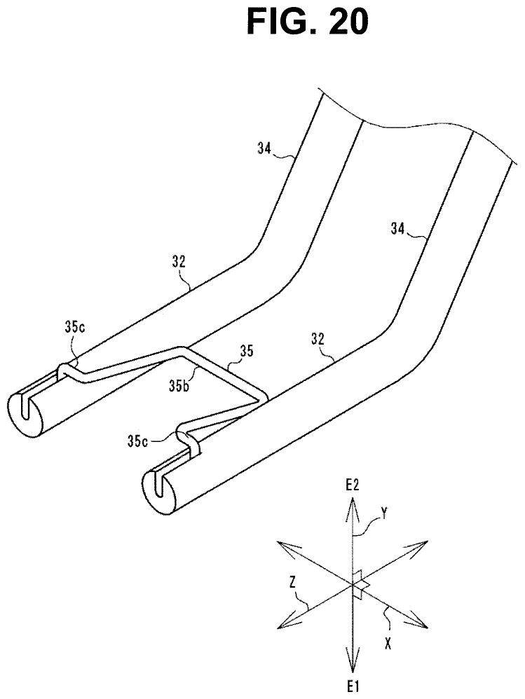

FIG. 20 shows a third modification of the exposed part 35c of the electrode unit 30 of the present embodiment. Exposed parts 35c of the third modification shown in FIG. 20 protrude in the second direction E2 from outer surfaces of the electrode support parts 32.

Each exposed part 35c protrudes in the direction opposite to the direction in which the electrode support part 32 is pushed against the wall surface 100a, each exposed part 35c protruding from the surface of the electrode support part 32 on a side opposite to the portion (the end 32c) which is pushed against the wall surface 100a of the organ 100. With the electrode unit 30 of the modification, the exposed parts 35c can be exposed to the inside of the organ 100 so as to come into contact with perfusate with certainty in a state where the electrode support parts 32 and the electrode center part 35b are caused to sink into the wall surface 100a of the organ 100.

In the electrode units 30 of the first to third modifications described heretofore, the exposed parts 35c are provided to the treatment electrode 35 as described above and hence, it is possible to prevent the entire treatment electrode 35 from sinking into the wall surface 100a of the organ 100. Therefore, with the electrode unit 30 and the resectoscope apparatus 10 of the first to third modifications, it is possible to prevent malfunction of a safety stop function of the high frequency power supply control device 55.

The configuration of the electrode unit 30 of the second embodiment including the above-described first to third modifications other than the exposed parts 35c is substantially equal to the configuration of the electrode unit 30 of the first embodiment. Therefore, with the electrode unit 30 and the resectoscope apparatus 10 of the present embodiment, the ends 32c of the two electrode support parts 32 in the first direction E1 are brought into contact with the tissue of the organ 100, thus maintaining a constant depth by which the electrode center part 35b sinks into the tissue. Accordingly, with the electrode unit 30 and the resectoscope apparatus 10 of the present embodiment, it is possible to easily control a depth of the tissue to be treated.

In the electrode unit 30 of the present embodiment including the first to third modifications, each arm part 34 may include the elastic support part 33 in the same manner as the electrode unit 30 of the second embodiment. In the case where the electrode unit 30 of the present embodiment includes the elastic support parts 33, as described in the second embodiment, the elastic support parts 33 are bent and hence, it is possible to maintain a substantially constant depth by which the electrode center part 35b sinks into the wall surface 100a even in a case where a force at or a trajectory along which the electrode support parts 32 are operated by the user fluctuates.

### Fourth Embodiment

Hereinafter, a fourth embodiment of the present invention will be described. Hereinafter, only points which make the fourth embodiment different from the first embodiment will be described. Constitutional elements substantially equal to the corresponding constitutional elements in the first embodiment are given the same reference symbols, and the description of such constitutional elements will be omitted when appropriate.

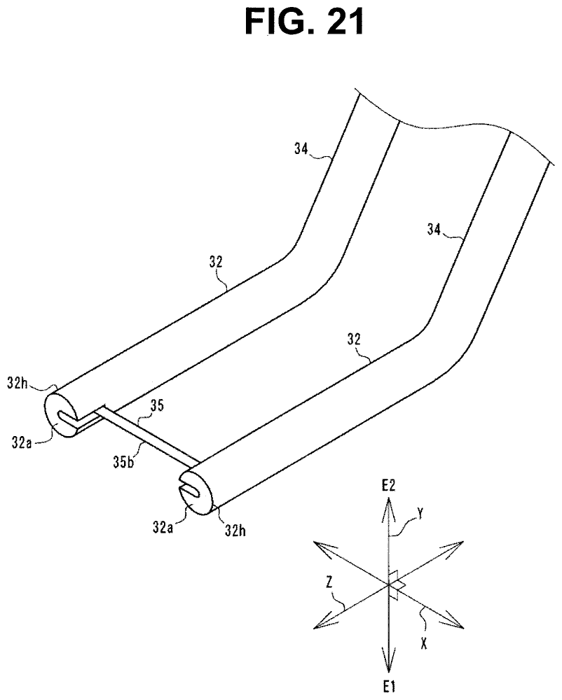

The fourth embodiment differs from the first embodiment with respect to the configuration of the electrode unit 30. FIG. 21 is a perspective view of electrode support parts 32 and a treatment electrode 35 of the electrode unit 30 of the fourth embodiment.

As shown in FIG. 21, in the electrode unit 30 of the present embodiment, the electrode center part 35b is disposed such that the entire electrode center part 35b is contained within profiles of the two electrode support parts 32 when viewed in the direction along the second axis X.

Specifically, each of the two electrode support parts 32 in the present embodiment includes a protruding part 32h that protrudes toward a distal end side from the electrode center part 35b. In other words, the electrode center part 35b is disposed on a proximal end side of the distal ends 32a of the two electrode support parts 32. A surface of each protruding part 32h is made of a material having electrical insulating property.

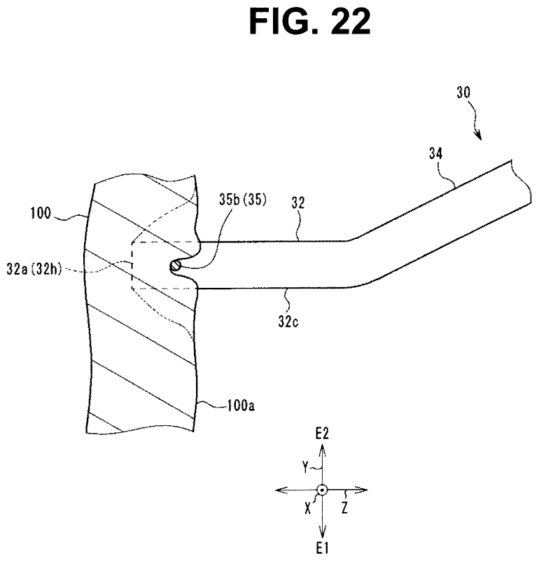

In a case of resecting tissue in the organ 100 by using the electrode unit 30 and the resectoscope apparatus 10 of the present embodiment, as shown in FIG. 22, the user can cause the electrode center part 35b to sink into the wall surface 100a by a predetermined depth by pushing the protruding parts 32h of the two electrode support parts 32 against the wall surface 100a of the organ 100.

In the present embodiment, when the distal ends 32a of the two electrode support parts 32 of the electrode unit 30 are pushed against the wall surface 100a of the organ 100, the protruding parts 32h serve as stoppers that restrict a depth by which the electrode center part 35b sinks into the wall surface 100a.

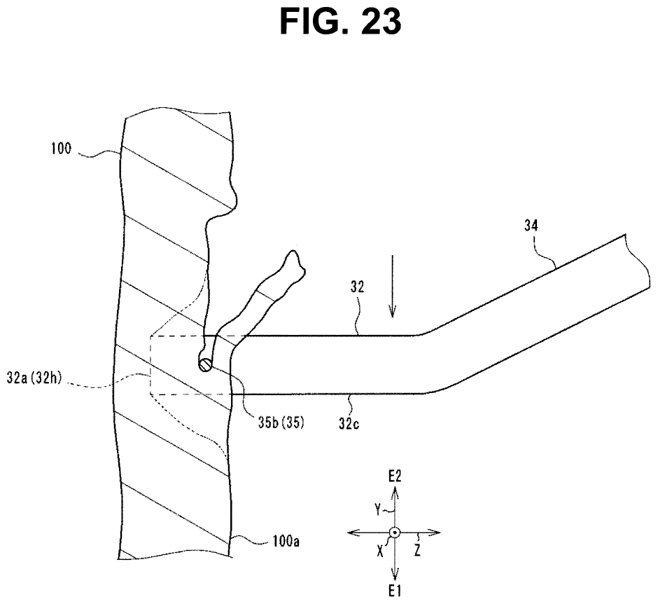

When the user moves the two electrode support parts 32 in the direction along the first axis Z while pushing the distal ends 32a of the two electrode support parts 32 against the wall surface 100a, as shown in FIG. 23, the tissue of the wall surface 100a of the organ 100 is cut out with a constant thickness.

Also in the electrode unit 30 of the present embodiment, in the same manner as the first embodiment, the ends 32c of the two electrode support parts 32 in the first direction E1 serve as stoppers that restrict a depth by which the electrode center part 35b sinks into the wall surface 100a.

That is to say, in performing treatment by using the electrode unit 30 of the present embodiment, according to an angle of the wall surface 100a with respect to the sheath 11 and the electrode unit 30, the user can selectively adopt a case where the ends 32c of the two electrode support parts 32 in the first direction E1 are pushed against the wall surface 100a or a case where the distal ends 32a of the two electrode support parts 32 are pushed against the wall surface 100a.

For example, in a case where the wall surface 100a is at an angle where the wall surface 100a extends along the sheath 11, treatment can be easily performed on the tissue of the wall surface 100a by pushing the ends 32c of the two electrode support parts 32 in the first direction E1 against the wall surface 100a. For example, in a case where the wall surface 100a has an angle at which the wall surface 100a is orthogonal to the sheath 11, treatment can be easily performed on the tissue of the wall surface 100a by pushing the distal ends 32a of the two electrode support parts 32 against the wall surface 100a.

With the electrode unit 30 and the resectoscope apparatus 10 of the present embodiment, even in a case where a posture of the sheath 11 in the organ 100 is limited, it is possible to perform treatment on the tissue of the wall surface 100a at various angles.

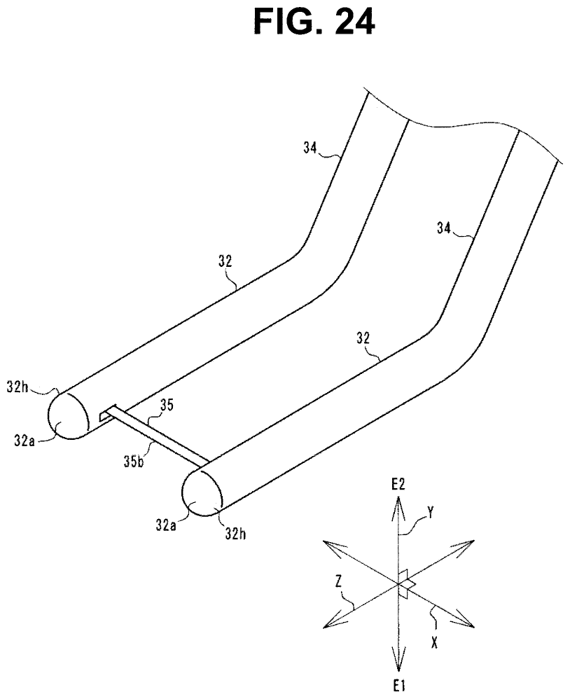

FIG. 24 shows a first modification of the protruding parts 32h of the electrode unit 30 of the present embodiment. The distal end 32a of each protruding part 32h of the first modification shown in FIG. 24 has a rounded shape. Specifically, a profile of the protruding part 32h of the modification has a hemispherical shape. The profile of the protruding part 32h of the modification is not limited to a hemispherical shape. For example, the profile of the protruding part 32h may be an ellipsoid or a paraboloid. Alternatively, the profile of the protruding part 32h may have a shape obtained by chamfering a corner portion of a cylinder with a predetermined radius.

With the electrode unit 30 of the modification, it is possible to prevent the two electrode support parts 32 from being caught by the tissue when the two electrode support parts 32 are moved in a state where the distal ends 32a of the two electrode support parts 32 are pushed against the wall surface 100a, so that the two electrode support parts 32 can be smoothly moved.

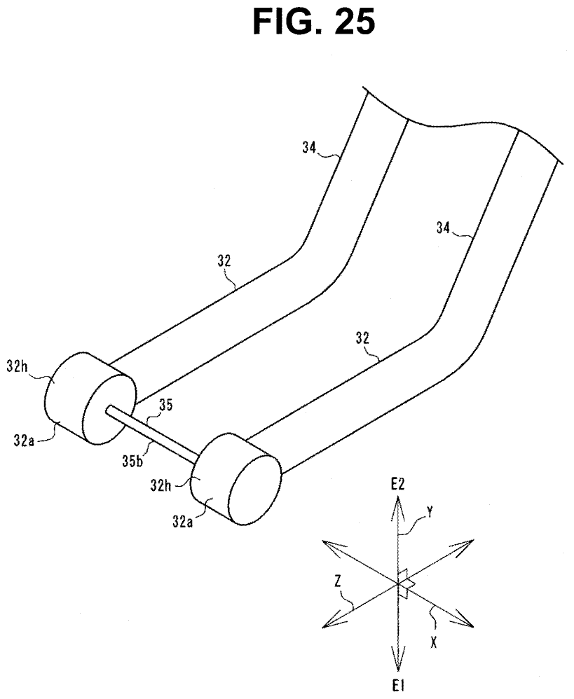

FIG. 25 shows a second modification of the protruding parts 32h of the electrode unit 30 of the present embodiment. In the second modification shown in FIG. 25, the electrode center part 35b has a straight line shape which is parallel to the second axis X. A profile of each protruding part 32h of the modification has a circular columnar shape with the electrode center part 35b being a center axis.

In the modification, a distance from the electrode center part 35b to an outer surface of each protruding part 32h is equal when viewed in the direction along the second axis X. Further, in a state where the protruding parts 32h are pushed against the wall surface 100a, a contact area between the protruding parts 32h and the wall surface 100a is substantially constant regardless of an angle of the electrode support parts 32 with respect to the wall surface 100a. Therefore, with the electrode unit 30 of the modification, it is possible to maintain a substantially constant depth by which the electrode center part 35b sinks into the wall surface 100a even in a case where an angle at which the user pushes the electrode support parts 32 against the wall surface 100a changes.

In the modification, the contact area between the electrode support parts 32 and the wall surface 100a is reduced compared with a case where the entire electrode support parts 32 are pushed against the wall surface 100a. Therefore, the electrode center part 35b can be caused to reach a predetermined depth in the wall surface 100a with a smaller force and hence, it is possible to suppress an amount of deformation of the electrode support parts 32 and the arm parts 34. When the amount of deformation of the electrode support parts 32 and the arm parts 34 is suppressed, the electrode center part 35b can be easily maintained in the field of view FOV of the telescope 21.

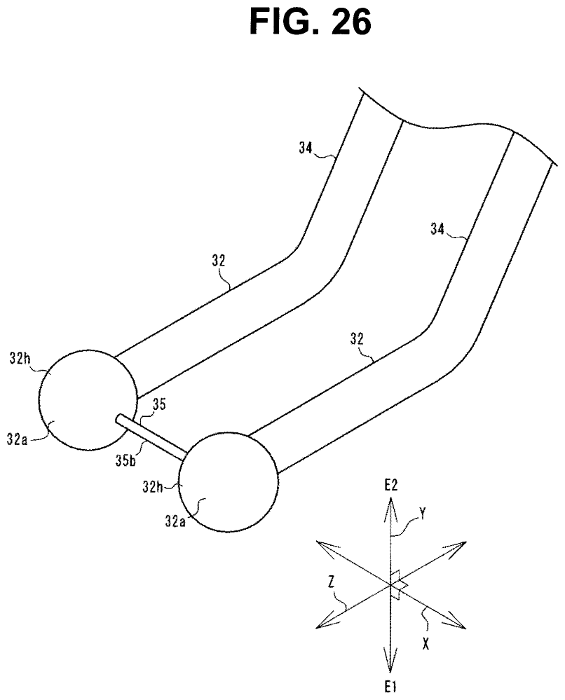

FIG. 26 shows a third modification of the protruding parts 32h of the electrode unit 30 of the present embodiment. In the third modification shown in FIG. 26, the electrode center part 35b has a straight line shape which is parallel to the second axis X. A profile of each protruding part 32h of the modification has a spherical shape. A center axis of the electrode center part 35b passes through the center of each protruding part 32h having a spherical shape.

In the modification, a distance from the electrode center part 35b to the outer surface of each protruding part 32h is equal when viewed in the direction along the second axis X. Further, in a state where the protruding parts 32h are pushed against the wall surface 100a, a contact area between the protruding parts 32h and the wall surface 100a is substantially constant regardless of an angle of the electrode support parts 32 with respect to the wall surface 100a. Therefore, with the electrode unit 30 of the modification, it is possible to maintain a substantially constant depth by which the electrode center part 35b sinks into the wall surface 100a even in a case where an angle at which the user pushes the electrode support parts 32 against the wall surface 100a changes.

In the modification, the contact area between the electrode support parts 32 and the wall surface 100a is reduced compared with a case where the entire electrode support parts 32 are pushed against the wall surface 100a. Therefore, the electrode center part 35b can be caused to reach a predetermined depth in the wall surface 100a with a smaller force and hence, it is possible to suppress an amount of deformation of the electrode support parts 32 and the arm parts 34. When the amount of deformation of the electrode support parts 32 and the arm parts 34 is suppressed, the electrode center part 35b can be easily maintained in the field of view FOV of the telescope 21.

The protruding parts 32h in the fourth embodiment shown in FIG. 23 and the protruding parts 32h of the modifications of the fourth embodiment shown in FIG. 24 to FIG. 26 may be made of an elastic material, such as rubber, having lower rigidity than the electrode support parts 32. By forming the protruding parts 32h by using an elastic material, it is possible to prevent damage to the tissue when the distal ends 32a of the two electrode support parts 32 are pushed against the wall surface 100a.

In the electrode unit 30 of the fourth embodiment including the above-described modifications, each arm part 34 may include the elastic support part 33 in the same manner as the electrode unit 30 of the second embodiment. In the case where the electrode unit 30 of the present embodiment includes the elastic support parts 33, as described in the second embodiment, the elastic support parts 33 are bent and hence, it is possible to maintain a substantially constant depth by which the electrode center part 35b sinks into the wall surface 100a even in a case where a force at or a trajectory along which the electrode support parts 32 are operated by the user fluctuates.

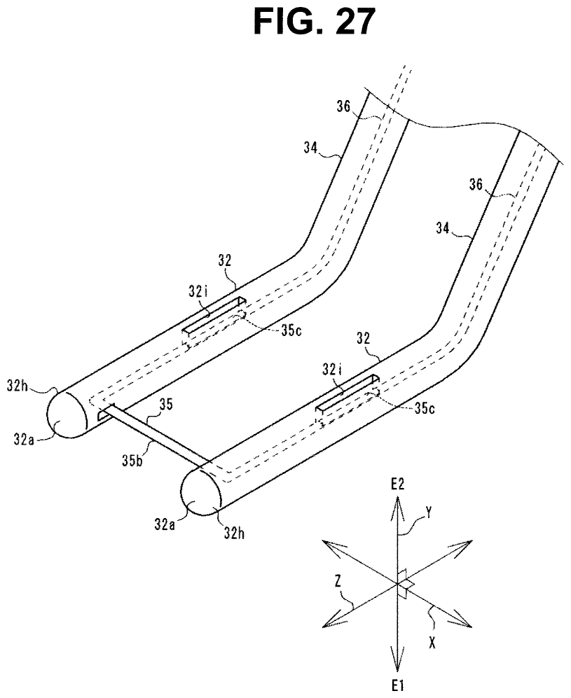

In the electrode unit 30 of the fourth embodiment including the modifications, the treatment electrode 35 may include the exposed part 35c in the same manner as the electrode unit 30 of the third embodiment. For example, as in the case of a fourth modification shown in FIG. 27, in the electrode unit 30 of the fourth embodiment, exposed parts 35c may be formed by providing window parts 32i, each of which is a hole extending to the wire 36 from a surface of the electrode support part 32 that faces in the second direction E2. The window part 32i may be provided to each arm part 34. As shown in FIG. 27, in the case where the treatment electrode 35 of the electrode unit 30 of the present embodiment includes the exposed parts 35c, as described in the third embodiment, the exposed parts 35c can be exposed to the inside of the organ 100 so as to come into contact with perfusate in a state where the electrode center part 35b is caused to sink into the wall surface 100a of the organ 100 and hence, it is possible to prevent malfunction of a safety stop function of the high frequency power supply control device 55.

### Fifth Embodiment

Hereinafter, a fifth embodiment of the present invention will be described. Hereinafter, only points which make the fifth embodiment different from the second embodiment will be described. Constitutional elements substantially equal to the corresponding constitutional elements in the second embodiment are given the same reference symbols, and the description of such constitutional elements will be omitted when appropriate.

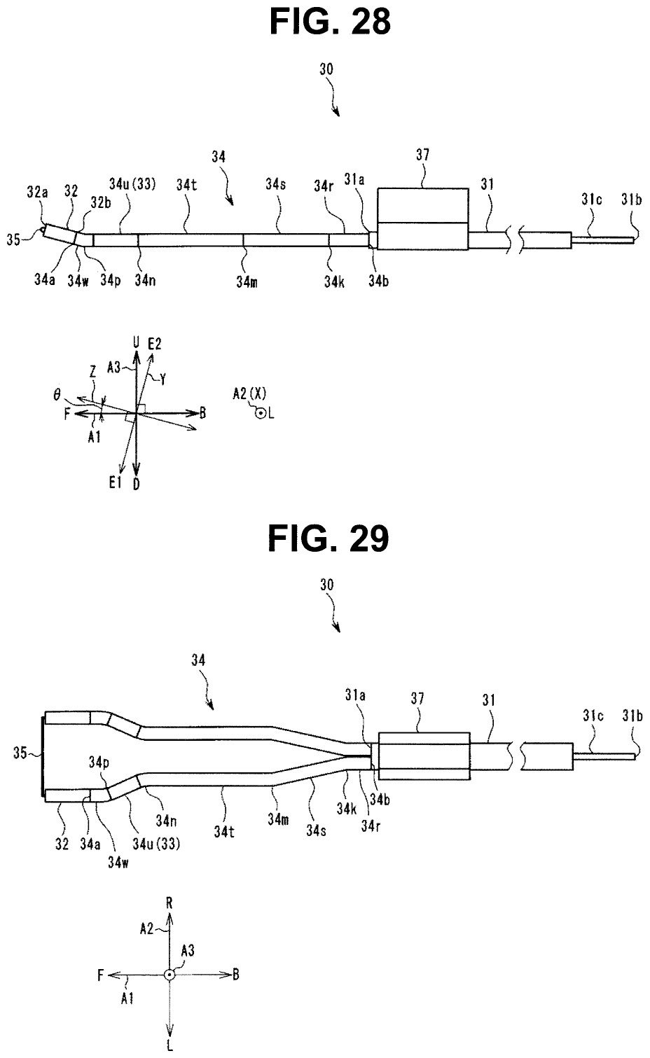

The fifth embodiment differs from the second embodiment with respect to a shape of the arm parts 34 of the electrode unit 30. FIG. 28 is a view of an electrode unit 30 of the fifth embodiment as viewed in a direction along the axis A2. FIG. 29 is a view of the electrode unit 30 of the fifth embodiment as viewed in a direction along the axis A3.

Each of two arm parts 34 in the present embodiment includes a first bent part 34k, a second bent part 34m, a third bent part 34n, and a fourth bent part 34p in this order from the proximal end 34b toward the distal end 34a. In the description made hereinafter, a portion of each of the two arm parts 34 which is disposed in the proximal end direction B with respect to the first bent part 34k is taken as a first section 34r. A portion of each of the two arm parts 34 which is disposed between the first bent part 34k and the second bent part 34m is taken as a second section 34s. A portion of each of the two arm parts 34 which is disposed between the second bent part 34m and the third bent part 34n is taken as a third section 34t. A portion of each of the two arm parts 34 which is disposed between the third bent part 34n and the fourth bent part 34p is taken as a fourth section 34u. A portion of each of the two arm parts 34 which is disposed in the distal end direction F with respect to the fourth bent part 34p is taken as a fifth section 34w. In the present embodiment, the first section 34r, the second section 34s, the third section 34t, the fourth section 34u, and the fifth section 34w of each of the two arm parts 34 have a straight line shape.

Two first sections 34r are fixed to the base part 31. The first sections 34r are disposed substantially parallel to the longitudinal axis AI. When viewed in the direction along the axis A3, two second sections 34s are disposed in such a manner that a distance by which the two second sections 34s are separated from each other in the direction along the axis A2 increases as the two second sections 34s extend in the distal end direction F. Further, the two second sections 34s are disposed parallel to a plane orthogonal to the axis A3.

Two third sections 34t are disposed parallel to the longitudinal axis A1. When viewed in the direction along the axis A3, two fourth sections 34u are disposed in such a manner that a distance by which the two fourth sections 34u are separated from each other in the direction along the axis A2 increases as the two fourth sections 34u extend in the distal end direction F. Further, the two fourth sections 34u are disposed parallel to the plane orthogonal to the axis A3. The fifth sections 34w are parallel to the first axis Z. That is to say, the fifth sections 34w are parallel to the electrode support parts 32. The fifth sections 34w are fixed to the electrode support parts 32.

In the present embodiment, the elastic support part 33 is provided to each of the two fourth sections 34u. The elastic support parts 33 have lower bending rigidity than the electrode support parts 32 and the base part 31.

With the electrode unit 30 and the resectoscope apparatus 10 of the present embodiment, an angle of the end 32c of each electrode support pan 32 in the first direction E1 can be changed due to curving deformation of each elastic support part 33 to conform to an angle of the wall surface 100a of the organ 100 with respect to the sheath 11. Therefore, with the electrode unit 30 and the resectoscope apparatus 10 of the present embodiment, the elastic support parts 33 are bent and hence, it is possible to maintain a substantially constant depth by which the electrode center part 35b sinks into the wall surface 100a even in a case where a force at or a trajectory along which the electrode support parts 32 are operated by the user fluctuates.

The present invention is not limited to the above-mentioned embodiments, and can be suitably changed without departing from the gist or concept of the invention which can be read in claims and in the entire description. An electrode unit and a resectoscope apparatus with such changes also fall within a technical scope of the present invention.

What is claimed is:

1. An electrode unit which performs treatment on tissue in a subject by using a high frequency current under observation with an endoscope, the electrode unit comprising:
two electrode support members disposed in a separated manner in a direction along a predetermined axis;
a base part extending along an axis intersecting with the predetermined axis, and having a distal end coupled with proximal ends of the two electrode support members; and
an electrode having both ends supported by the two electrode support members, wherein
the electrode includes an exposed portion in a region which overlaps with at least one of the two electrode support members when viewed from a second direction which is opposite to a first direction in which the two electrode support members are pushed against the subject during use of the electrode unit, the exposed portion being exposed only in the second direction.

2. The electrode unit according to claim 1, further comprising:
an elastic support member disposed between each of the proximal ends of the two electrode support members and the distal end of the base part, and having lower bending rigidity than the two electrode support members and the base part.

3. The electrode unit according to claim 1, wherein
the exposed portion protrudes from an outer surface of at least one of the two electrode support members in the second direction.

4. An electrode unit which performs treatment on tissue in a subject by using a high frequency current under observation with an endoscope, the electrode unit comprising:
two electrode support members disposed in a separated manner in a direction along a predetermined axis;
a base part extending along an axis intersecting with the predetermined axis, and having a distal end coupled with proximal ends of the two electrode support members; and
an electrode having both ends supported by the two electrode support members, wherein
the electrode includes an electrode center part disposed so as to be contained within profiles of the two electrode support members when viewed from a distal end side of the two electrode support members.

5. The electrode unit according to claim 4, wherein
a distal end of each of the two electrode support members has a rounded shape.

6. The electrode unit according to claim 4, wherein
a distal end of each of the two electrode support members is made of an elastic material.

7. The electrode unit according to claim 2, wherein
the two electrode support members are inclined with respect to a longitudinal axis of the base part at a predetermined angle.

8. The electrode unit according to claim 7, further comprising:
two arm parts coupled with proximal ends of the two electrode support members, and coupled with a distal end of the base part, wherein
each of the two arm parts includes:
a first bent portion which is bent so as to extend in a second direction as the first bent portion extends from a proximal end toward a distal end, the second direction being opposite to a first direction in which the two electrode support members are pushed against the subject during the use of the electrode unit;
a second bent portion which is provided on a distal end side of the first bent portion, and which is bent so as to extend in the first direction as the second bent portion extends toward the distal end; and
a third bent portion which is provided on a distal end side of the second bent portion and on a first direction side of the distal end of the base part, and which is bent to the predetermined angle so as to extend in the second direction as the third bent portion extends toward the distal end, and
the elastic support member is disposed between the second bent portion and the third bent portion of each of the two arm parts.

9. A resectoscope apparatus comprising:
a sheath inserted into a subject and having a hollow shape;
a telescope including an insertion portion inserted into the sheath;
an electrode unit holding part configured to hold the electrode unit according to claim 4 in the sheath; and
a scope holding part configured to hold the insertion portion of the telescope in the sheath at a position in a second direction with respect to the two electrode support members of the electrode unit, the second direction being opposite to a first direction in which the two electrode support members are pushed against the subject during use of the electrode unit.
